(19) United States
(12) Patent Application Publication
(10) Pub. No.: US 2026/0153512 A1
(43) Pub. Date: Jun. 4, 2026

(54) ESTIMATING GLOMERULAR FILTRATION RATES IN FELINES

(71) Applicant: Société des Produits Nestlé S.A., vevey (CH)

(72) Inventor: Qinghong LI, Chesterfield, MO (US)

(73) Assignee: Société des Produits Nestlé S.A., vevey (CH)

(21) Appl. No.: 19/299,108

(22) Filed: Aug. 13, 2025

Related U.S. Application Data
(60) Provisional application No. 63/727,581, filed on Dec. 3, 2024.

(51) Int. Cl. G01N 33/68; G01N 33/50

(52) U.S. Cl. CPC G01N 33/6812; G01N 33/50; G01N 2650/00; G01N 2800/347

(57) ABSTRACT

A method of estimating glomerular filtration rate of a feline can include measuring a blood sample of a feline for blood serum concentrations of pseudouridine and acetylthreonine, and optionally phenylacetylglutamine, and/or tryptophan, and also calculating a blood serum level product. The method can also include applying a feline factor as a multiple to the blood serum level product resulting in an estimated glomerular filtration rate. Calculating the blood serum level product can be based on a first weighted value based upon the blood serum concentration of pseudouridine, a second weighted value based upon the blood serum concentration of acetylthreonine,, an optional third weighted value based upon the blood serum concentration of phenylacetylglutamine, and an optional fourth weighted value based upon the blood serum concentration of tryptophan.

### CROSS REFERENCE TO RELATED APPLICATION

This application claims priority to U.S. Provisional Application Ser. No. 63/727,581 filed Dec. 3, 2024 the disclosure of which is incorporated in its entirety herein by this reference.

### BACKGROUND

Renal/urinary health in cats, including diagnosis of renal diseases such as chronic kidney disease (CKD), is an area in feline healthcare that has presented challenges, particularly as it relates to early diagnosis. Approximately 30% of cats over the age of 10 may be affected by some type of renal disease, and early detection may be a challenge. As an example, CKD is typically diagnosed based on progressive changes in parameters in blood chemistry and urinalysis, including blood serum creatinine, symmetric dimethylarginine (SDMA), urine specific gravity, and urine protein to creatine ratio. Creatinine concentration can be measured using enzymatic reactions that can be detected using the spectrophotometric assay, but in many instances, large inter-assay differences often exist, particularly in the lower concentration ranges where the inaccuracies are more pronounced. Furthermore, regarding SDMA assays, bias and imprecision may be problematic in some instances.

As an example, stage (1) one CKD can be especially challenging to discover because blood serum levels of creatinine and SDMA are typically rising within a reference interval, resulting in a proper diagnosis of CKD to include multiple blood samplings and evaluations at different points in time. As a result, there are many cats that do not ever receive a proper diagnosis of CKD during the early stages (stages 1 or 2), and by the time the disease progresses to a more advanced stage, unrecoverable kidney damage may have already occurred.

In further detail, though measuring the glomerular filtration rate (GFR) in the cat may be a good approach for diagnosing renal impairment, current techniques are not typically feasible for routine veterinary practice. Thus, unless measuring glomerular filtration rate directly, other estimation methods tend to lack the sensitivity, objectivity, and specificity for reliably detecting CKD. In accordance with this, practical improvements in the way that CKD may be diagnosed (particularly early-stage CKD) that is reasonably accurate and more accessible to cat owners or veterinarians would be desirable.

### BRIEF DESCRIPTION OF THE DRAWINGS

FIG. 1 is a flow diagram illustrating an example method of estimating glomerular filtration rate of felines in accordance with the present disclosure.

FIG. 2 is a flow diagram illustrating example methods of diagnosing and treating chronic kidney disease of felines in accordance with the present disclosure.

FIG. 3 illustrates a plot of data collected from example experiments where estimated glomerular filtration rates (eGFR) of cats were determined based on a product of weighted blood serum concentrations of four (4) metabolites multiplied by a feline factor in accordance with the present disclosure.

### DETAILED DESCRIPTION

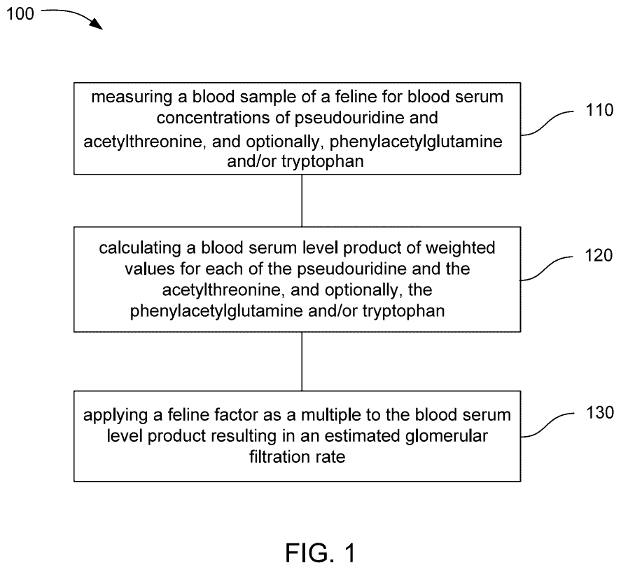

In accordance with examples of the present disclosure, a method of estimating glomerular filtration rate of a feline is shown by way of example in FIG. 1 at 100, and can include measuring 110 a blood sample of a feline for blood serum concentrations of pseudouridine and acetylthreonine, and optionally phenylacetylglutamine and/or tryptophan, and also calculating 120 a blood serum level product. The method can also include applying 130 a feline factor as a multiple to the blood serum level product resulting in an estimated glomerular filtration rate. Calculating the blood serum level product can be based on a first weighted value based upon the blood serum concentration of pseudouridine, a second weighted value based upon the blood serum concentration of acetylthreonine, an optional third weighted value based upon the blood serum concentration of phenylacetylglutamine, and/or an optional fourth weighted value based on the blood serum concentration tryptophan.

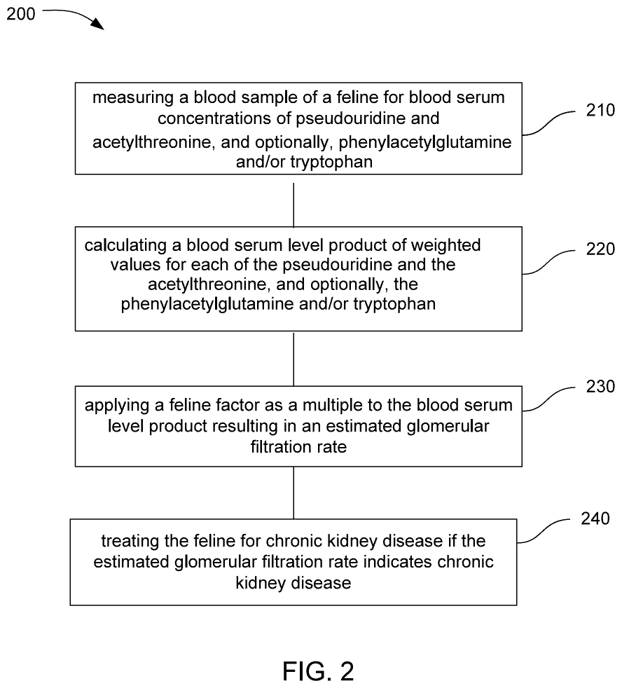

In another example, a method of diagnosing and treating chronic kidney disease (CKD) of a feline is shown by way of example in FIG. 2 at 200, and can include measuring 210 a blood sample of a feline for blood serum concentrations of pseudouridine and acetylthreonine, and optionally phenylacetylglutamine and/or tryptophan, and also calculating 220 a blood serum level product. The blood serum level product can be based on a first weighted value based upon the blood serum concentration of pseudouridine, a second weighted value based upon the blood serum concentration of acetylthreonine, an optional third weighted value based upon the blood serum concentration of phenylacetylglutamine, and/or an optional a fourth weighted value based on the blood serum concentration tryptophan. The method can also include applying 230 a feline factor as a multiple to the blood serum level product resulting in an estimated glomerular filtration rate, and treating 240 the feline for CKD if the estimated glomerular filtration rate indicates CKD.

It is noted that when discussing examples and embodiments related to the various systems and methods herein, such discussions can be considered applicable to one another whether or not they are explicitly discussed in the context of that example or embodiment. Thus, for example, when discussing methods of estimating glomerular filtration rates of a feline or diagnosing and treating CKD of a feline in the context of these methods, disclosure from one method is applicable to the other method.

Furthermore, terms used herein will have their ordinary meaning in the relevant technical field unless specified otherwise. In some instances, there are terms defined more specifically throughout the specification, with a few more general terms included at the end of the specification.

Additional features and advantages of the disclosed method and apparatus are described in, and will be apparent from, the following Detailed Description and the Figures.

The features and advantages described herein are not all-inclusive and, in particular, many additional features and advantages will be apparent to one of ordinary skill in the art in view of the figures and description. Moreover, it should be noted that the language used in the specification has been principally selected for readability and instructional purposes, and not to limit the scope of the inventive subject matter.

Chronic kidney disease (CKD) in feline animals, such as cats, can be difficult to diagnose, particularly in the early stages, e.g., stages 1 or 2, where obvious symptoms may not be manifested. State of the art diagnosis and staging (for stages 1-4) often relies on clinical presentation and physical examination, and staging often relies on testing serum levels of creatinine and symmetric dimethylarginine (SDMA), and urine specific gravity and urinary protein/creatinine ratio. The glomerular filtration rate (GFR) is regarded as the gold standard for assessing renal function. Measuring GFR is a complex process that requires intravenous administration of clearance markers followed by multiple samplings, making it time-consuming, labor-intensive, and stressful for both pets and their owners. Approximately 75% of nephrons are nonfunctioning before serum creatinine levels rise above the reference range. Although blood creatinine is widely recognized as a standard surrogate marker for cGFR, its production can be influenced by non-renal factors such as muscle mass. Body weight loss is commonly observed in cats with CKD and occurs prior to the formal diagnosis of CKD, with a median loss of 11% over a 12-month period. On average, serum SDMA becomes increased seventeen months before creatinine. However, the assertion that SDMA is superior to creatinine as a reliable marker for GFR changes was not confirmed. Point-of-care SDMA measurements also showed poor correlations with GFR. Additional diagnostic tools and tests that can aid in the early detection of kidney dysfunction are crucial for effective management and treatment of feline CKD.

With challenges and some difficulties associated with using creatinine and SMDA as a proxy for GFR, it has been found that the GFR can be effectively estimated in a more accessible manner for obtaining an eGFR.

Specifically, the present inventor has found a process of estimating glomerular filtration rate, which is robust, effective, and practical. In one embodiment, estimating glomerular filtration rate of a feline can include measuring a blood sample of a feline for blood serum concentrations of pseudouridine and acetylthreonine, and optionally phenylacetylglutamine and/or tryptophan, and also calculating a blood serum level product. The method can also include applying a feline factor as a multiple to the blood serum level product resulting in an estimated glomerular filtration rate. Calculating the blood serum level product can be based on a first weighted value based upon the blood serum concentration of pseudouridine, a second weighted value based upon the blood serum concentration of acetylthreonine, an optional third weighted value based upon the blood serum concentration of phenylacetylglutamine, and/or an optional fourth weighted value based on the blood serum concentration tryptophan.

In some embodiments, the methods can include diagnosing and treating CKD of a feline. In this example, in addition to utilizing the methods of estimating glomerular filtration rate of a feline, methods can further include treating the feline for CKD if the estimated glomerular filtration rate indicates CKD. For example, a method of diagnosing chronic kidney disease in a feline can comprising measuring the estimated glomerular filtration rate (eGFR) as described herein; and determining that the feline has chronic kidney disease when the eGFR is 1.15 mL/min/kg or less, that the feline has early-stage chronic kidney disease when the eGFR is from 0.5 mL/min/kg to 1.15 mL/min/kg, and/or that the feline has late-stage chronic kidney disease when the eGFR is less than 0.5 ml/min/kg.

The methods can further include treating the feline, for example, by modifying the feline diet to include a wet pet food for improved kidney function. In some examples, modifying the feline diet may include feeding the cat a wet pet food for improved kidney function with no more than about 0.45 weight percentage (wt %) phosphorus content and no more than a 0.35 wt % sodium content (at all stages of CKD). If the CKD is late stage (stage 3 or stage 4), the wet pet food can further be restricted to no more than about 28 wt % protein content. In some examples, modification of the feline diet may include supplementing the food with omega-3 fatty acids and/or prebiotic fiber. Other diets that may help improve kidney function can also be selected for use.

Regarding obtaining the eGFR, this value can be determined, for example, by measuring the blood serum levels of two (2) metabolites, weighting those blood serum level measurements, and applying a feline factor (f) that has been derived specifically for estimating the renal health of feline animals, e.g., cats. The two metabolites that can be used in this manner include pseudouridine, and acetylthreonine. An example formula for estimating the glomerular filtration rate of feline animals is provided in Formula 1, as follows:

$$eGFR = f * [ {( W )}^{p} {( Y )}^{r} ] \qquad Formula 1$$

where:
eGFR is the estimated glomerular filtration rate,
f is the feline factor,
W is the blood serum concentration of pseudouridine,
Y is the blood serum concentration of acetylthreonine,
p provides a first weight to the first weighted value for pseudouridine, and
r provides a second weight to the second weighted value for acetylthreonine.

In another embodiment, the eGFR can be determined by measuring the blood serum levels of three (3) metabolites, weighting those blood serum level measurements, and applying a feline factor (f) that has been derived specifically for estimating the renal health of feline animals, e.g., cats. The first two metabolites that can be used in this manner include pseudouridine, and acetylthreonine and the third metabolite can be either phenylacetylglutamine or tryptophan. Example formulas for estimating the glomerular filtration rate of feline animals are provided in Formulas 2 and 3, as follows:

$$eGFR ≅ {f}^{*} [ {( W )}^{p} {( X )}^{q} {( Y )}^{r} ] \qquad Formula 2$$

$$eGFR ≅ {f}^{*} [ {( W )}^{p} {( Y )}^{r} {( Z )}^{s} ] \qquad Formula 3$$

where:
eGFR is the estimated glomerular filtration rate,
f is the feline factor,
W is the blood serum concentration of pseudouridine,
X is the blood serum concentration of phenylacetylglutamine,
Y is the blood serum concentration of acetylthreonine,
Z is the blood serum concentration of tryptophan,
p provides a first weight to the first weighted value for pseudouridine,
r provides a second weight to the second weighted value for acetylthreonine, and
q provides a third weight to the third weighted value for phenylacetylglutamine (Formula 2),
or
s provides a third weight to the third weighted value for tryptophan (Formula 3).

In another embodiment, the eGFR can be determined by measuring the blood serum levels of all four (4) metabolites, weighting those blood serum level measurements, and applying a feline factor (f) that has been derived specifically for estimating the renal health of feline animals, e.g., cats. The four metabolites that can be used in this manner include pseudouridine, phenylacetylglutamine, acetylthreonine, and tryptophan. An example formula for estimating the glomerular filtration rate of feline animals is provided in Formula 4, as follows:

$$eGFR ≅ {f}^{*} [ {( W )}^{p} {( Y )}^{r} {( X )}^{q} {( Z )}^{s} ] \qquad Formula 4$$

where
eGFR is the estimated glomerular filtration rate,
f is the feline factor,
W is the blood serum concentration of pseudouridine,
Y is the blood serum concentration of acetylthreonine,
X is the blood serum concentration of phenylacetylglutamine,
Z is the blood serum concentration of tryptophan,
p provides a first weight to the first weighted value for pseudouridine,
r provides a second weight to the second weighted value for acetylthreonine,
q provides a third weight to the third weighted value for phenylacetylglutamine, and
s provides a fourth weight to the fourth weighted value for tryptophan.

Generally, the feline factor (f) can be from about 0.3 to about 0.425. In specific aspects, the feline factor (f) can be from about 0.312 to about 0.406, from about 0.325 to about 0.375, from about 0.354 to about 0.365, or about 0.359. Generally, p can be from about −0.9 to about −0.7. In specific aspects, p can be from about −0.85 to about −0.75, from about −0.825 to about −0.775, or about −0.805. Generally, q can be from about −0.05 to about −0.04. In specific aspects, q can be from about −0.0485 to about −0.0435, from about-0.0475 to about −0.045, or about −0.047. Generally, r can be from about −0.08 to about −0.07. In specific aspects, r can be from about −0.0785 to about −0.0715, from about −0.0775 to about −0.0725, or about −0.075. Generally, s can be from about 0.26 to about 0.27. In specific aspects, s can be from about 0.25 to about 0.275, from about 0.26 to about 0.27, from about 0.265 to about 0.2675, or about 0.266.

In accordance with this, using a combination of these example weighted exponential values (p, q, r, and/or s) with a combination of at least two of the four above-identified metabolites, and by applying the feline factor (f), a reasonably accurate estimate of renal function in cats can be made, which is sensitive enough to catch even early stage CKD, e.g., stages 1 or 2. In this example, the blood serum concentrations of pseudouridine and acetylthreonine, and optionally phenylacetylglutamine and tryptophan can be measured in micrograms per milliliter (μg/mL) and the eGFR of early stage (stage 1 or stage 2) CKD in felines can be from about 0.5 mL/min/kg to about 1.15 mL/min/kg. Notably, in accordance with the present disclosure, stage 1 and stage 2 CKD is considered to be early-stage CKD, even though the IRIS Guidelines include only some stage 2 cats as exhibiting early onset CKD.

In further detail regarding the feline factor (f), for example, this multiple can be determined by selecting a value that when multiplied with the blood serum level product, accurately predicts the presence of at least early stage CKD, e.g., stages 1 or 2, in alignment with an established protocol in the veterinarian space for staging CKD in cats. For example, the 2023 IRIS Guidelines can be used for the comparison. Essentially, the IRIS Guidelines set forth established protocols in the veterinarian space for diagnosing, staging, and treating CKD in dogs and cats. With specific respect to the IRIS Guidelines for cats, stage 1 and early stage 2 CKD can be established by the following: 1) one or both diagnostic findings of creatinine or symmetric dimethylarginine (SDMA) increasing within a reference interval (with no apparent prerenal cause); 2) persistent increase in SDMA (>14 μg/dL); 3) abnormal kidney imaging; and persistent renal proteinuria (UPC >0.4). Regarding late stage 2 to stage 4 CKD, this may be determined by one or more of the following: 1) increased blood serum levels in both creatinine and SDMA; and 2) modified urine specific gravity (<1.035). Regarding staging (or establishing the correct stage of the disease), creatinine and SDMA blood serum levels can be used to more specifically establish the four generally accepted stages of CKD, based on the following in Table 1:

TABLE 1
IRIS Guidelines for CKD for Felines

| | Stage 1 Blood | Stage 2 Blood | Stage 3 Blood | Stage 4 Blood |
|---|---|---|---|---|
| Compound | Serum Levels | Serum Levels | Serum levels | Serum Levels |
| Creatinine | <1.6 mg/dL | 1.6-2.8 mg/dL | 2.9-5.0 mg/dL | >5.0 mg/dL |
| | (140 μmol/L) | (140-250 μmol/L) | (251-440 μmol/L) | (440 μmol/L) |
| SDMA | <18 μg/dL | 18-25 μg/dL | 26-38 μg/dL | >38 μg/dL |

Stage 1 (no azotemia and normal creatinine);
Stage 2 (mild azotemia and normal or mildly elevated creatinine);
Stage 3 (moderate azotemia);
Stage 4 (severe azotemia)

In accordance with this, when selecting an appropriate feline factor, use of this established protocol for staging chronic kidney disease (CKD) can include obtaining blood serum concentrations for any of the data provided in Table 1. In some examples, as the present disclosure is very helpful in establishing early stage CKD, the following values can be used for comparison purposes in calculating a reasonable range of values for the feline factor (f): creatinine at less than about 1.6 mg/dL and SDMA at less than about 18 μg/dL to indicate stage 1 CKD; and/or creatinine at from about 1.6-2.8 mg/dL and SDMA at from about 18-25 μg/dL to indicate stage 2 CKD.

Thus, the methods of estimating GFR in felines described herein can provide a reasonably simple way diagnosing (and treating as needed) chronic kidney disease (CKD), particularly in the early stages, e.g., stage 1 or 2, where it can be difficult in some instances to otherwise make such a diagnosis. The use of these metabolites for the estimate can be particularly useful in the early stages, as creatinine does not have to be measured. As creatinine at early-stage CKD is not particularly useful for a stage 1 diagnosis, the fact that other compounds can be measured to reliably estimate glomerular filtration is one benefit provided by the present disclosure. Furthermore, blood concentrations of these metabolites described herein can be relatively simple to determine using well-known lab techniques, making estimating glomerular filtration in this manner relatively simple and reliable for evaluating whether a cat may be suffering from early onset CKD.

### Example Embodiments

In accordance with the disclosure herein, the following examples are illustrative of several embodiments of the present technology.
1. A method of estimating glomerular filtration rate of a feline, the method comprising:
measuring a blood sample of a feline for blood serum concentrations of pseudouridine and acetylthreonine;
calculating a blood serum level product of a first weighted value based upon the blood serum concentration of pseudouridine and a second weighted value based upon the blood serum concentration of acetylthreonine; and
applying a feline factor as a multiple to the blood serum level product resulting in an estimated glomerular filtration rate.
2. The method of example 1, wherein the feline factor is determined by selecting a value that when multiplied with the blood serum level product, accurately predicts the presence of at least early stage chronic kidney disease in alignment with an established protocol in the veterinarian space for staging chronic kidney disease in cats, wherein the established protocol for staging chronic kidney disease includes blood serum concentrations for: creatinine at less than about 1.6 mg/dL and symmetric dimethylarginine at less than about 18 μg/dL to indicate stage 1 chronic kidney disease, creatinine at from about 1.6-2.8 mg/dL and symmetric dimethylarginine at from about 18-25 μg/dL to indicate stage 2 chronic kidney disease, or a combination thereof.
3. The method of example 1, wherein a formula for estimating the glomerular filtration rate is represented by Formula 1:

$$eGFR ≅ {f}^{*} [ ( W ) p ( Y ) r ] \qquad Formula 1$$

wherein:
eGFR is the estimated glomerular filtration rate,
f is the feline factor,
W is the blood serum concentration of pseudouridine,
Y is the blood serum concentration of acetylthreonine,
p provides a first weight to the first weighted value for pseudouridine, and
r provides a second weight to the second weighted value for acetylthreonine.
4. The method of example 3, wherein f is from about 0.3 to about 0.425, p is from about −0.9 to about −0.7, and r is from about −0.08 to about −0.07.
5. The method of example 1, further comprising measuring phenylacetylglutamine and calculating a blood serum level product of a third weighted value based upon the blood serum concentration of phenylacetylglutamine.
6. The method of example 5, wherein a formula for estimating the glomerular filtration rate is represented by Formula 2:

$$eGFR ≅ {f}^{*} [ ( W ) p ( Y ) r ( X ) q ] \qquad Formula 2$$

wherein:
eGFR is the estimated glomerular filtration rate,
f is the feline factor,
W is the blood serum concentration of pseudouridine,
Y is the blood serum concentration of acetylthreonine,
X is the blood serum concentration of phenylacetylglutamine,
p provides a first weight to the first weighted value for pseudouridine,
r provides a second weight to the second weighted value for acetylthreonine, and
q provides a third weight to the third weighted value for phenylacetylglutamine.
7. The method of example 6, wherein f is from about 0.3 to about 0.425, p is from about −0.9 to about −0.7, r is from about −0.08 to about −0.07, and q is from about 0.05 to about 0.04.
8. The method of example 1, further comprising measuring tryptophan and calculating a blood serum level product of a third weighted value based upon the blood serum concentration of tryptophan.
9. The method of example 8, wherein a formula for estimating the glomerular filtration rate is represented by Formula 3:

$$eGFR ≅ {f}^{*} [ ( W ) p ( Y ) r ( Z ) s ] \qquad Formula 3$$

wherein:
eGFR is the estimated glomerular filtration rate,
f is the feline factor,
W is the blood serum concentration of pseudouridine,
Y is the blood serum concentration of acetylthreonine,
Z is the blood concentration of tryptophan,
p provides a first weight to the first weighted value for pseudouridine,
r provides a second weight to the second weighted value for acetylthreonine, and
s provides a third weight to the third weighted value for tryptophan.
10. The method of example 9, wherein f is from about 0.3 to about 0.425, p is from about −0.9 to about −0.7, r is from about −0.08 to about −0.07, and s is from about 0.26 to about 0.27.
11. The method of example 1, further comprising measuring phenylacetylglutamine and tryptophan, calculating a blood serum level product of a third weighted value based upon the blood serum concentration of phenylacetylglutamine, and calculating a blood serum level product of a fourth weighted value based upon the blood serum concentration of tryptophan.
12. The method of example 11, wherein a formula for estimating the glomerular filtration rate is represented by Formula 4:

$$eGFR ≅ {f}^{*} [ ( W ) p ( Y ) r ( X ) q ( Z ) s ] \qquad Formula 4$$

wherein:
eGFR is the estimated glomerular filtration rate,
f is the feline factor,
W is the blood serum concentration of pseudouridine,
Y is the blood serum concentration of acetylthreonine,
X is the blood serum concentration of phenylacetylglutamine,
Z is the blood serum concentration of tryptophan,
p provides a first weight to the first weighted value for pseudouridine,
r provides a second weight to the second weighted value for acetylthreonine,
q provides a third weight to the third weighted value for phenylacetylglutamine, and
s provides a fourth weight to the fourth weighted value for tryptophan.
13. The method of example 12, wherein f is from about 0.3 to about 0.425, p is from about −0.9 to about −0.7, q is from about −0.05 to about −0.04, r is from about −0.08 to about-0.07, and s is from about 0.26 to about 0.27.
14. A method of diagnosing chronic kidney disease in a feline, comprising:
measuring the estimated glomerular filtration rate (eGFR) of example 1 or any preceding example; and
determining that the feline has chronic kidney disease when the eGFR is 1.15 mL/min/kg or less, that the feline has early-stage chronic kidney disease when the eGFR is from 0.5 mL/min/kg to 1.15 mL/min/kg; and/or that the feline has late-stage chronic kidney disease when the eGFR is less than 0.5 mL/min/kg.
15. A method of diagnosing and treating chronic kidney disease in a feline, comprising:
measuring the estimated glomerular filtration rate (eGFR) of example 1 or any preceding example;
determining that the feline has chronic kidney disease when the eGFR is 1.15 mL/min/kg or less, that the feline has early-stage chronic kidney disease when the eGFR is from 0.5 mL/min/kg to 1.15 mL/min/kg; and/or that the feline has late-stage chronic kidney disease when the eGFR is less than 0.5 mL/min/kg; and
treating the feline for chronic kidney disease if the eGFR indicates chronic kidney disease.
16. The method of example 15, wherein treating the feline includes modifying the feline diet to include a wet pet food for improved kidney function with no more than about 0.45 wt % phosphorus content and no more than a 0.35 wt % sodium content, and wherein when the chronic kidney disease is late stage chronic kidney disease, the wet pet food is further restricted to no more than about 28 wt % protein content.

### Definitions

As used herein, “about,” “approximately” and “substantially” are understood to refer to numbers in a range of numerals, for example the range of −10% to +10% of the referenced number, −5% to +5% of the referenced number, −1% to +1% of the referenced number, or −0.1% to +0.1% of the referenced number. All numerical ranges herein should be understood to include all integers, whole or fractions, within the range. Moreover, these numerical ranges should be construed as providing support for a claim directed to any number or subset of numbers in that range. For example, a disclosure of from 1 to 10 should be construed as supporting a range of from 1 to 8, from 3 to 7, from 1 to 9, from 3.6 to 4.6, from 3.5 to 9.9, and so forth.

As used in this disclosure and the appended claims, the singular forms “a,” “an” and “the” include plural referents unless the context clearly dictates otherwise. Thus, for example, reference to “a component” or “the component” includes two or more components.

The words “comprise,” “comprises” and “comprising” are to be interpreted inclusively rather than exclusively. Likewise, the terms “include,” “including” and “or” should all be construed to be inclusive, unless such a construction is clearly prohibited from the context. Thus, a disclosure of an embodiment using the term “comprising” includes a disclosure of embodiments “consisting essentially of” and “consisting of” the components identified.

The term “and/or” used in the context of “X and/or Y” should be interpreted as “X,” or “Y,” or “X and Y.” Similarly, “at least one of X or Y” should be interpreted as “X,” or “Y,” or “X and Y.”

Where used herein, the terms “example,” “embodiment,” “such as,” etc., particularly when followed by a listing of terms, are merely exemplary and illustrative and should not be deemed to be exclusive or comprehensive.

Although the present disclosure has been described in certain specific aspects, many additional modifications and variations would be apparent to those skilled in the art. In particular, any of the various processes described above can be performed in alternative sequences and/or in parallel (on the same or on different computing devices) in order to achieve similar results in a manner that is more appropriate to the requirements of a specific application. It is therefore to be understood that the present disclosure can be practiced otherwise than specifically described without departing from the scope and spirit of the present disclosure. Thus, aspects of the present disclosure should be considered in all respects as illustrative and not restrictive. It will be evident to the annotator skilled in the art to freely combine several or all of the aspects discussed here as deemed suitable for a specific application of the disclosure. Throughout this disclosure, terms like “improved”, “example(s)” etc. indicate elements or dimensions which are particularly suitable (but not essential) to the disclosure or an embodiment thereof, and may be modified wherever deemed suitable by the skilled annotator, except where expressly required. Accordingly, the scope of the present disclosure should be determined not by the embodiments illustrated, but by the appended claims and their equivalents.

While the present disclosure has been described above with particularity and detail in connection with what is presently deemed to be the most practical embodiments of the disclosure, it will be apparent to those of ordinary skill in the art that numerous modifications, including, but not limited to, variations in size, materials, shape, form, function and manner of operation, assembly and use may be made without departing from the principles and concepts set forth herein.

### Examples

The following examples illustrate embodiments of the disclosure that are presently best known. However, it is to be understood that the following are only exemplary or illustrative of the application of the principles of the present technology. Numerous modifications and alternative compositions, methods, and systems may be devised by those skilled in the art without departing from the spirit and scope of the present disclosure. The appended claims are intended to cover such modifications and arrangements. Thus, while the present disclosure has been described herein with particularity, the following examples provide further detail in connection with what are presently deemed to be practical embodiments of the disclosure.

### Example 1-Chronic Kidney Disease (CKD) Diagnoses and Staging of Cat Populations

CKD diagnoses and staging was performed on 34 cats according to the 2023 IRIS Guidelines set forth in Table 1 above. Specifically, using these guidelines, clinical presentation and physical examinations were conducted, and staging was based primarily on creatinine and symmetric dimethylarginine (SDMA) blood serum levels to determine which of the four (4) generally accepted stages of CKD was present. Based on these criteria, it was established that the 34 cat population included 14 healthy cats (non-CKD cats), 14 cats with earlier stage CKD (stages 1 or early stage 2), including 2 stage 1 CKD cats; and 6 cats with later stage CKD (stages 3 or 4).

### Example 2-Blood Serum Collection and Blood Serum Measurements of four (4) Metabolites

With the known disease states of the 34 cat population (14 healthy, 14 early stage CKD, and 6 late stage CKD), blood was collected from the various cats for quantification of pseudouridine, phenylacetylglutamine, acetylthreonine, and tryptophan, and the blood serum levels were analyzed by Liquid Chromatography with tandem mass spectrometry (LC-MS/MS). Cat blood serum samples were spiked with internal standards and subjected to protein precipitation and then centrifuged. Following centrifugation, an aliquot of the supernatant was injected onto an Agilent 1290/AB SCIEX QTRAP 5500 LC-MS/MS system equipped with an amide Ultra Performance Liquid Chromatography (UPLC) column. The mass spectrometer was operated in positive Hydrophilic Interaction Liquid Chromatography (HILIC) mode for tryptophan and phenylacetylglutamine and negative HILIC mode for pseudouridine, both using electrospray ionization (ESI). Acetylthreonine was analyzed in both HILIC modes. The peak area of the product ions was measured against the peak area of the respective internal standard product ions. Quantitation was performed using a weighted linear least squares regression analysis generated from calibration standards prepared immediately prior to each run. LC-MS/MS raw data was collected and processed using SCIEX software Analyst 1.6.3. Data reduction was performed using Microsoft Excel for Office 365 v.16.

### Example 3-Calculating Estimated Glomerular Filtration Rate (eGFR) of Cat Population

The glomerular filtration rates of the cats across the population were estimated in accordance with Formula 4 to obtain the eGFR (mL/min/kg) for the 34 cats in the population to determine if the estimates were the same as the diagnoses and staging as established using the ISIS Guidelines outlined in Example 1. In accordance with Formula 4, for this specific example, the following values were used: f is about 0.359, W is the blood serum level of pseudouridine (μg/mL), Y is the blood serum level of acetylthreonine (μg/mL), X is the blood serum level of phenylacetylglutamine (μg/mL), Z is the blood serum level of tryptophan, p is about −0.805 (μg/mL), r is about −0.075, q is about −0.047, and s is about 0.266, which is expressed below as Formula 5, as follows:

$$eGFR ≅ {0.359}^{*} [ {( W )}^{- 0.805} {( Y )}^{- 0.075} {( X )}^{- 0.047} {( Z )}^{0266} ] \qquad Formula 5$$

Table 2 below includes the data collected for the full population of cats (34 total cats) based on a healthy control group of cats (non-CKD), early stage CKD cats (stage 1 or stage 2), and late stage CKD cats (stage 3 or stage 4), with a control group of cats being healthy (non-CKD), as follows:

TABLE 2
Estimated Glomerular Filtration Rates (eGFR) of Cat Populations

| | Control Cats | | |
|---|---|---|---|
| | (non-CKD) | Stage 1 or 2 CKD | Stage 3 or 4 CKD |
| Range of eGFR | >=1.15 | 0.5-1.15 | <0.5 |
| (ml/min/kg) | | | |
| eGFR Mean ± SE | 1.37 ± 0.05 | 0.78 ± 0.06 | 0.36 ± 0.06 |
| (mL/min/kg) | | | |

### Example 4-Comparative Methods of Estimating Glomerular Filtration Rates

For comparative purposes, an alternative method of estimating the glomerular filtration rates (eGFR) in cats was studied, which included estimating GFR using creatinine and symmetric dimethylarginine (SDMA) levels in accordance with more conventional approaches. For this comparative, GFR was determined for 34 cats (14 healthy, 14 early stage CKD, and 6 late stage CKD) using creatinine blood serum levels (mg/dL) and SDMA blood serum levels (μg/dL) with p and q values (the exponent values) in accordance with Formula 3, as follows:

$$GFR ≅ {( Creatinine )}^{- 1.5} {( SDMA )}^{- 0.25} \qquad Formula 6$$

### Example 5-Comparison of eGFR Accuracy using Formula 5 (Example 3) vs. eGFR Accuracy using Comparative Formula 6 (Example 4)

Data collected using the eGFR performance data of Formula 5 (Example 3) and the GFR comparative performance data of Formula 6 (Example 4) is provided in Table 3, as follows:

TABLE 3
Comparison of eGFR (Example 3) and
GFR of Comparative Example 4

| | Estimate Methodology | | |
|---|---|---|---|
| | Control Cats | | |
| | (non-CKD) | Stage 1 or 2 CKD | Stage 3 or 4 CKD |
| Formula 5 | 96 | 66.7% | 85.7% |
| Formula 6 | 64 | 60% | 100% |

Percentages relate to cats that are correctly diagnosed and staged.

As can be seen in Table 3, the early-stage estimates using the methodology of the present disclosure outperformed the early-stage estimates by a more conventional estimation methodology. The late-stage performance was still acceptable, though it did not perform quite as well as the comparative methodology. However, late-stage CKD is not nearly as difficult to identify, as at stages 3 and 4, the cat will typically experience symptoms that would be much more apparent to a cat owner, for example. It is the early-stage CKD that is difficult to discover. Early diagnosis can lead to early treatment before any serious kidney damage has occurred.

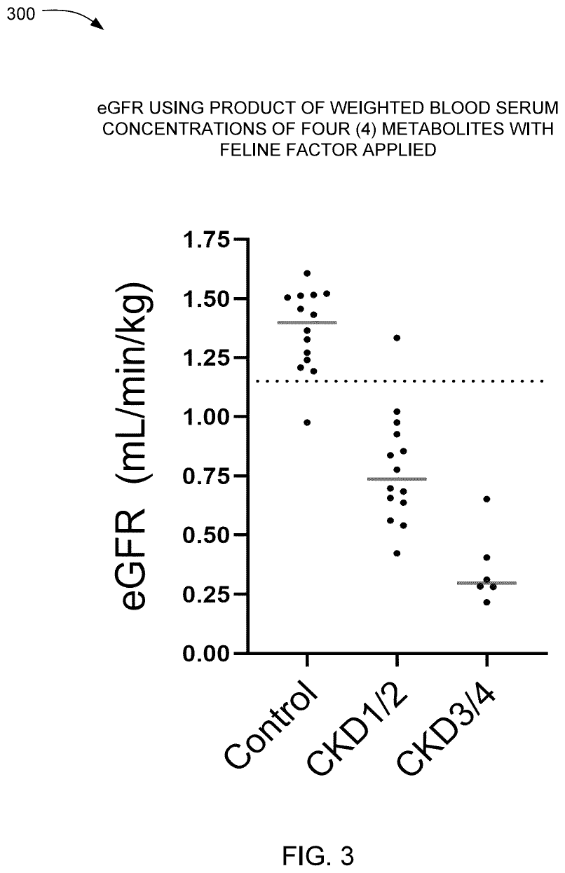

For additional details regarding the estimate data collected using the Formula 4 equation (or the more specific Formula 5 equation), FIG. 3 is provided by way of example illustrating the estimated glomerular filtration rate (eGFR) using cat blood serum concentrations of the four identified metabolites (pseudouridine, phenylacetylglutamine, acetylthreonine, and tryptophan), the weighted (exponent) values, and the feline factor (f). Each cat from each population is shown in FIG. 3 individually along with a short horizontal line indicating the median value (mL/min/kg). Dashed line at 1.15 (mL/min/kg) indicate example lines of demarcation separating values where healthy cats and early-stage CKD cats may reside relative to their metabolite levels as applied to Formula 4 (or Formula 5 in this specific example).

What is claimed is:

1. A method of estimating glomerular filtration rate of a feline, the method comprising:
measuring a blood sample of a feline for blood serum concentrations of pseudouridine and acetylthreonine;
calculating a blood serum level product of a first weighted value based upon the blood serum concentration of pseudouridine and a second weighted value based upon the blood serum concentration of acetylthreonine; and
applying a feline factor as a multiple to the blood serum level product resulting in an estimated glomerular filtration rate.

2. The method of claim 1, wherein the feline factor is determined by selecting a value that when multiplied with the blood serum level product, accurately predicts the presence of at least early stage chronic kidney disease in alignment with an established protocol in the veterinarian space for staging chronic kidney disease in cats, wherein the established protocol for staging chronic kidney disease includes blood serum concentrations for: creatinine at less than about 1.6 mg/dL and symmetric dimethylarginine at less than about 18 μg/dL to indicate stage 1 chronic kidney disease, creatinine at from about 1.6-2.8 mg/dL and symmetric dimethylarginine at from about 18-25 μg/dL to indicate stage 2 chronic kidney disease, or a combination thereof.

3. The method of claim 1, wherein a formula for estimating the glomerular filtration rate is represented by Formula 1:
$$eGFR ≅ {f}^{*} [ {( W )}^{p} {( Y )}^{r} ] \qquad Formula 1$$
wherein:
eGFR is the estimated glomerular filtration rate,
f is the feline factor,
W is the blood serum concentration of pseudouridine,
Y is the blood serum concentration of acetylthreonine,
p provides a first weight to the first weighted value for pseudouridine, and
r provides a second weight to the second weighted value for acetylthreonine.

4. The method of claim 3, wherein f is from about 0.3 to about 0.425, p is from about −0.9 to about −0.7, and r is from about −0.08 to about −0.07.

5. The method of claim 1, further comprising measuring phenylacetylglutamine and calculating a blood serum level product of a third weighted value based upon the blood serum concentration of phenylacetylglutamine.

6. The method of claim 5, wherein a formula for estimating the glomerular filtration rate is represented by Formula 2:
$$eGFR ≅ {f}^{*} [ {( W )}^{p} {( Y )}^{r} {( X )}^{q} ] \qquad Formula 2$$
wherein:
eGFR is the estimated glomerular filtration rate,
f is the feline factor,
W is the blood serum concentration of pseudouridine,
Y is the blood serum concentration of acetylthreonine,
X is the blood serum concentration of phenylacetylglutamine,
p provides a first weight to the first weighted value for pseudouridine,
r provides a second weight to the second weighted value for acetylthreonine, and
q provides a third weight to the third weighted value for phenylacetylglutamine.

7. The method of claim 6, wherein f is from about 0.3 to about 0.425, p is from about −0.9 to about −0.7, r is from about −0.08 to about −0.07, and q is from about 0.05 to about 0.04.

8. The method of claim 1, further comprising measuring tryptophan and calculating a blood serum level product of a third weighted value based upon the blood serum concentration of tryptophan.

9. The method of claim 8, wherein a formula for estimating the glomerular filtration rate is represented by Formula 3:
$$eGFR ≅ {f}^{*} [ {( W )}^{p} {( Y )}^{r} {( Z )}^{s} ] \qquad Formula 3$$
wherein:
eGFR is the estimated glomerular filtration rate,
f is the feline factor,
W is the blood serum concentration of pseudouridine,
Y is the blood serum concentration of acetylthreonine,
Z is the blood concentration of tryptophan,
p provides a first weight to the first weighted value for pseudouridine,
r provides a second weight to the second weighted value for acetylthreonine, and
s provides a third weight to the third weighted value for tryptophan.

10. The method of claim 9, wherein f is from about 0.3 to about 0.425, p is from about −0.9 to about −0.7, r is from about −0.08 to about −0.07, and s is from about 0.26 to about 0.27.

11. The method of claim 1, further comprising measuring phenylacetylglutamine and tryptophan, calculating a blood serum level product of a third weighted value based upon the blood serum concentration of phenylacetylglutamine, and calculating a blood serum level product of a fourth weighted value based upon the blood serum concentration of tryptophan.

12. The method of claim 11, wherein a formula for estimating the glomerular filtration rate is represented by Formula 4:
$$eGFR ≅ {f}^{*} [ {( W )}^{p} {( Y )}^{r} {( X )}^{q} {( Z )}^{s} ] \qquad Formula 4$$
wherein:
eGFR is the estimated glomerular filtration rate,
f is the feline factor,
W is the blood serum concentration of pseudouridine,
Y is the blood serum concentration of acetylthreonine,
X is the blood serum concentration of phenylacetylglutamine,
Z is the blood serum concentration of tryptophan,
p provides a first weight to the first weighted value for pseudouridine,
r provides a second weight to the second weighted value for acetylthreonine,
q provides a third weight to the third weighted value for phenylacetylglutamine, and
s provides a fourth weight to the fourth weighted value for tryptophan.

13. The method of claim 12, wherein f is from about 0.3 to about 0.425, p is from about −0.9 to about −0.7, q is from about −0.05 to about −0.04, r is from about −0.08 to about-0.07, and s is from about 0.26 to about 0.27.

14. A method of diagnosing chronic kidney disease in a feline, comprising:
measuring the estimated glomerular filtration rate (eGFR) of claim 1; and
determining that the feline has chronic kidney disease when the eGFR is 1.15 mL/min/kg or less, that the feline has early-stage chronic kidney disease when the eGFR is from 0.5 mL/min/kg to 1.15 mL/min/kg; and/or that the feline has late-stage chronic kidney disease when the eGFR is less than 0.5 mL/min/kg.

15. A method of diagnosing and treating chronic kidney disease in a feline, comprising:
measuring the estimated glomerular filtration rate (eGFR) of claim 1;
determining that the feline has chronic kidney disease when the eGFR is 1.15 mL/min/kg or less, that the feline has early-stage chronic kidney disease when the eGFR is from 0.5 mL/min/kg to 1.15 mL/min/kg; and/or that the feline has late-stage chronic kidney disease when the eGFR is less than 0.5 mL/min/kg; and
treating the feline for chronic kidney disease if the eGFR indicates chronic kidney disease.

16. The method of claim 15, wherein treating the feline includes modifying the feline diet to include a wet pet food for improved kidney function with no more than about 0.45 wt % phosphorus content and no more than a 0.35 wt % sodium content, and wherein when the chronic kidney disease is late stage chronic kidney disease, the wet pet food is further restricted to no more than about 28 wt % protein content.
